(19) United States
(12) Patent Application Publication
(10) Pub. No.: US 2007/0083272 A1
(43) Pub. Date: Apr. 12, 2007

(54) Prosthetic knee joint

(75) Inventors: Paul G. Van De Veen, Enschede (NL); Hans Dietl, Gablitz (AT); Richard Skiera, Vienna (AT)

Correspondence Address: GREENBLUM & BERNSTEIN, P.L.C., 1950 ROLAND CLARKE PLACE, RESTON, VA, 20191, US

(73) Assignee: OTTO BOCK HEALTHCARE PRODUCTS GMBH, Vienna (AT)

(21) Appl. No.: 10/595,720

(22) PCT Filed: Nov. 4, 2004
(86) PCT No.: PCT/DE04/02456

§ 371 Date: Aug. 8, 2006

(30) Foreign Application Priority Data
Nov. 7, 2003 (DE) 103 51 916.5

(51) Int. Cl. A61F 2/62

(52) U.S. Cl. 623/39; 623/24

(57) ABSTRACT

The invention relates to a prosthetic knee-joint comprising an upper part with a fixing device for a receptacle of a leg stump and a lower part that is pivotally connected to the upper part by a multi-axial articulation device. The lower part can be straightened at all times in an unhindered manner and a locking device is provided to prevent flexion of the articulation device. The aim of the invention is to provide a prosthetic knee-joint that allows ease of movement when standing up and sitting down on a chair. In addition, said prosthetic knee-joint should remain stable and locked when standing and walking to provide maximum safety for the geriatric patient. To achieve this, the articulation device has a resistance element, which during a flexion exerts a resistance in opposition to the latter within a pre-definable angular range and which can be freely straightened at all times.

The invention relates to a prosthetic knee joint, with an upper part which has a fastening device for a receptacle for a leg stump, and with a lower part which is pivotably connected to the upper part via an articulation device. The invention relates in particular to a geriatric prosthetic knee joint, geriatric patients usually being understood as elderly persons who have lost much of their physical capacities and who usually have reduced kinesthesia and reduced mental powers.

There are presently many prosthetic knee joints on the market which are suitable for geriatric patients. For the patient group in question here, all prosthetic knee joints follow the same concept, namely a simple connection of upper part and lower part without means for controlling the swing phase, except for the friction that is always present, and with a mechanical lock which automatically locks the knee joint in the extended position. By operation of a release cable, the knee joint is unlocked and permits sitting in a flexed position of the prosthetic knee joint, the relation of the receptacle for the leg stump with respect to the artificial lower leg generally being 90°.

A disadvantage of the above-described concept is the fact that the patient can place a load on the prosthesis only when it is fully extended and locked. When the patient is sitting down, the energy required for standing up has to be exerted through the muscles of the healthy leg, assisted to a greater or lesser extent by the shoulder and arm muscles if walking aids or armrests can be used for getting up. Sufficient stability is present only when the movement is completed, that is to say when the person has stood up from the seated position and the leg is fully extended.

A further disadvantage is that, when the knee joint is unlocked, it can become immediately unstable, since the knee joint no longer affords any resistance. Controlled transfer to the seated position also requires powerful use of the healthy leg or of the leg muscles, which are not especially strong specifically in elderly patients. In practice, this means that the patients fall back to a greater or lesser extent into a seated position.

This has the consequence that standing up from a seated position is very difficult and strenuous, and sitting down is dangerous and entails considerable risks. As a result of this, many patients reduce the frequency with which they stand up and sit down to an absolute minimum, which leads to lack of movement, and this is detrimental to their general physical condition.

It is therefore necessary to offer the prosthesis user a prosthesis with which, on the one hand, he can safely walk about and stand and with which, on the other hand, he is ensured unrestricted sitting down and a corresponding mobility when seated. In addition, the transfer from standing to sitting, and vice versa, is a critical procedure which entails increased risk for the prosthesis user, since quickly dropping when sitting down, or falling back when standing up, can lead to falls and thus to injuries.

The object of the present invention is to make available a prosthetic knee joint which ensures improved safety of the prosthesis user. The disadvantages outlined above are to be eliminated, and the knee joint is intended to make it easy to stand up and safe to sit down on a chair. Moreover, the knee joint is to be stable and must be able to be locked during standing and walking in order to allow the geriatric patient the maximum degree of safety.

According to the invention, this object is achieved in principle by the fact that a movement is delayed, in extreme cases blocked, over a defined angle range, so that no uncontrolled sitting movement can arise and so that falling back into the seated position is avoided. In construction terms, this object is achieved by the fact that the articulation device has a resistance device which acts as a lock and which blocks a flexion (pivoting of the lower part counter to the normal walking direction) within a definable angle range, the lower part being freely pivotable in the flexion direction outside the definable angle range.

The resistance device is thus designed as an aid to standing up which, within the definable angle range, prevents the flexion of the articulation device. When standing up, the knee joint can be extended without having to work against an appreciable resistance, but the resistance device which works as a locking device continuously blocks a flexion movement, so that the prosthesis user is able to stand up in stages from the seated position, without running the risk of falling back into the seated position. Instead, the prosthesis user is able to stand up in a gradual manner. At any time, and in each angle position, the resistance device, which prevents pivoting back, can also be switched, so that, with a suitable angle of the knee joint, it is possible to switch to the operating mode with increased resistance, which permits a gentle lowering of the body into the seated position. The resistance device is thus designed to be switched, meaning that the resistance can be reduced and can also be increased to the level of a locking action. Delaying the falling back movement to zero ensures that the prosthesis user does not drop abruptly and in an uncontrolled manner onto the chair or to the ground.

In an embodiment of the prosthetic knee joint as a lockable knee joint, a catch device is provided which locks the articulation in the extended position. A catch device for forming a lockable knee joint is generally a mechanical catch, although other constructions of a catch are conceivable by which the prosthetic knee joint is locked in the extended position, so that the prosthesis user, in particular the geriatric prosthesis user, can safely stand and walk about. The catch device can be switched only between the “locked” and “released” states; no regulating or intermediate stages of the resistance are provided. The catch device is unlocked via an operating device and locked automatically or manually. The resistance device provided in addition to the catch device ensures that when the prosthesis user sits down, he is transferred gently from the standing position to the seated position, without the stability abruptly being lost when the knee joint is unlocked. During standing and walking, the knee joint can be blocked by the catch device, if appropriate assisted by the resistance device, and affords sufficient stability. As soon as the locking is released by the prosthesis user, the resistance device and thus the knee joint exerts an adjustable, high level of resistance against a flexion movement, so that the transfer to a seated position takes place gently, in a controlled manner and over a longer period of time. In the seated position, this high level of resistance, which is exerted over a definable angle range, is automatically reduced or cancelled out, and the knee joint can, over a small angle range, execute the flexion and extension movements that are usually made in the seated position.

It is provided that the catch device or the resistance device and locking device can be operated via an operating device in each angle position of the lower part relative to the upper part, which means that in each position of the knee joint a flexion movement is prevented by the locking device, but the extension movement, that is to say the pivoting of the lower leg in the walking direction, is still possible. It is likewise provided that the catch device and the resistance device can be switched in every position, that is to say released or reduced in resistance, so that a prosthesis user in each phase of standing up or sitting down can actively reduce the resistance or locking in order to get into the seated position.

In a further development of the invention, the operating device for locking or unlocking the locking device is operated manually or by motor. In order to be able to activate the operating device from a location remote from the knee, it has a cable which is coupled to the slide, to the rotary member or to a lever. This cable can be routed along the thigh through the clothing and can be secured in a relatively inconspicuous manner on a trouser waistband or inside a trouser pocket.

As an alternative to manual operation, provision is made that the operating device comprises a motor, an energy accumulator, a gear, and a control unit which is connected to a switch mechanism by remote control. Thus, at the press of a button or by remote control, the operating device can move a slide along a front articulation lever and effect the locking or resistance adjustment, so that manual operation of the operating device in the area of the knee joint is no longer necessary. This is advantageous especially for persons who cannot readily reach the knee joint. The manual module can be replaced by a motor-driven module, since the outer dimensions and the mechanical couplings with levers and the like are preferably compatible. The remote control and the operating device for the resistance device or locking device or also in combination with the catch device can be applied to all lockable knee joints and be used in combination with these. Remote controls are in particular infrared, radio or acoustic remote controls, but not a so-called “satellite switch”, that is to say a mechanical switch coupled to the respective device via a cable or Bowden wire.

A delay element, which is assigned to the operating device or the resistance device or locking device, allows the prosthesis user first to unlock the knee joint in a secured position and, after a predeterminable period of time, ensures that it returns to the locked position if the prosthesis user has not sat down within this period of time. This avoids a situation where a geriatric patient who has forgotten the unlocking or who has been distracted finds himself standing on an unlocked, easily movable knee joint without being aware of this. Such a state can lead to serious injuries if the prosthesis user tries to make a walking movement and bends the knee in the unlocked state.

It is also provided that the delay element ensures that the knee joint remains locked over a predeterminable period of time or remains acted upon by a high level of resistance. After the unlocking, the prosthesis user can then, for example, stand up with a stable knee or can use the hand that was used for the operation to support himself before the flexion of the knee joint is initiated. As delay element, it is possible to use electronic devices such as delay circuits for motors or valves; it is likewise possible to provide relays, elastic elements with rheological properties, and circuits with actuators or mechanical delay elements.

To be able to switch the resistance device as a function of the angle of the upper part relative to the lower part, a control device, preferably a mechanical control device, is provided which is connected to the upper part and which is coupled to the resistance device. In this way, it is possible, by simple means, to ensure that a suitable resistance is made available within a predetermined angle range.

The resistance device is advantageously designed as a hydraulic or pneumatic unit, a friction coupling or an electromagnetic coupling, in order to make available an adjustable resistance.

In one embodiment of the invention involving a hydraulic or pneumatic unit, a controllable valve system is provided which is arranged inside a piston guided in a cylinder. The piston forms part of the resistance device. A particularly compact structure is afforded by the combination and arrangement of the valve system inside the piston, and, for economical production of the prosthetic knee joint, the valve system is connected to the mechanical control device which, as a function of the angle of the upper part relative to the lower part, switches the resistance device such that an increased resistance is provided over a defined angle range. When this angle range is exceeded, there is no longer any resistance against further flexion in the walking direction; a flexion can be prevented at any time by corresponding locking means, whereas unimpeded extension counter to the walking direction is possible at all times.

This control device is arranged around the rotation axis formed by the upper part and by the piston rod secured thereon, resulting in a particularly simple arrangement of the control device relative to the piston rod and to the control rod guided therein.

The control device has a first cam disk which acts on the control rod and is connected in a rotationally fixed manner to the upper part or alternatively is entrained via a carrier, which is secured on the upper part, the first cam disk being switched via the carrier or carriers depending on the angle. If the first cam disk is mounted rotatably on the upper part at least one carrier acting in the flexion direction is needed which, when a certain flexion angle is reached, turns the cam disk such that a valve is opened, so that a free movement of the knee joint without resistance is possible.

A prosthetic knee joint, with an upper part which has a fastening device for a receptacle for a leg stump, and with a lower part which is pivotably connected to the upper part via an articulation device, and with a catch device for arresting the prosthetic knee joint in the extended position, the catch device being able to be locked and unlocked by an operating device, comprises, for ease of handling, an operating device which is triggered by remote control. The remote control and the operating device for the catch device can be applied to all lockable knee joints and can be used in combination with these. The remote controls are cableless remote controls, in particular infrared, radio or acoustic remote controls, but not a so-called “satellite switch”, that is to say a switch coupled to the respective device via a cable.

Independently of the design of a lockable knee joint with a resistance device, the catch device is assigned, according to the invention, a delay element which unlocks or re-locks the catch device after a time delay after activation of the unlocking. The delay element can be designed as a relay, as an elastic or rheological element or as an electronic circuit with actuator.

An illustrative embodiment of the invention is explained in more detail below with reference to the attached figures. Identical reference numbers in different figures designate identical structural elements. For reasons of clarity, not all structural parts are provided with reference numbers in all of the figures.

FIG. 1 shows a side view of a prosthetic knee joint according to the invention, with a fitted receptacle for the thigh, and with an artificial lower leg;

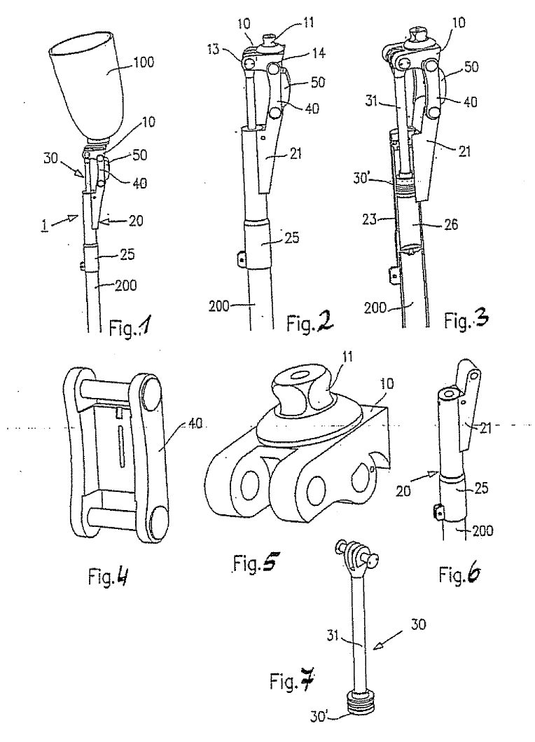

FIG. 2 shows an enlarged view of FIG. 1 without the receptacle for the thigh;

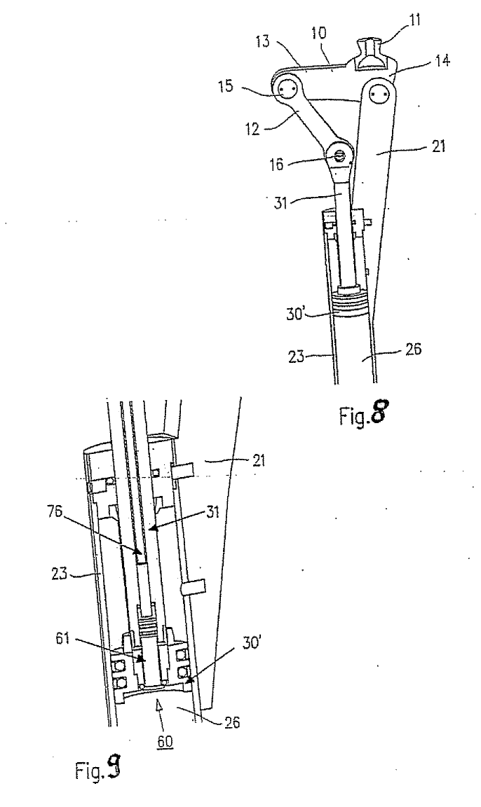

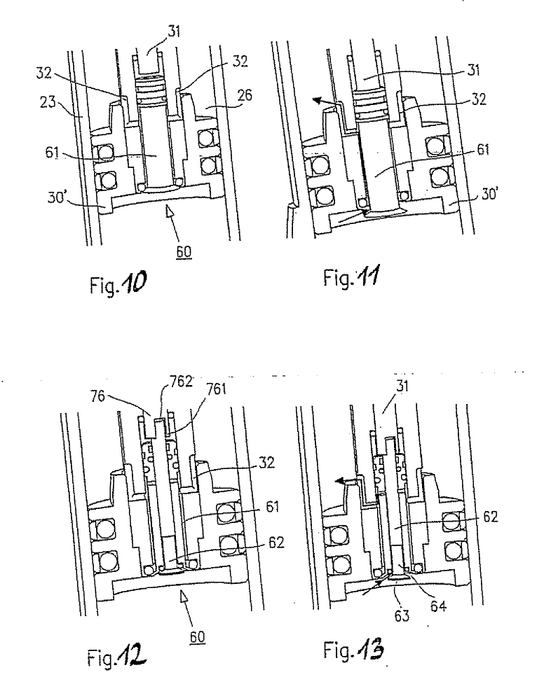

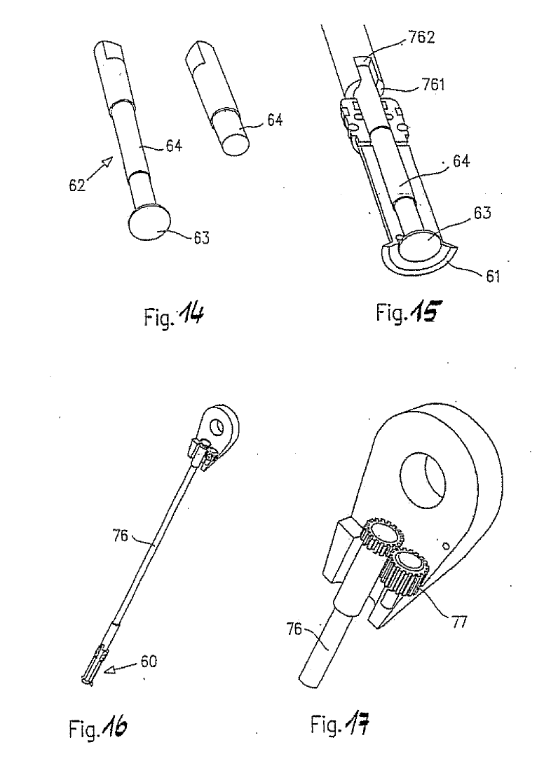

FIG. 3 shows a partial cross-sectional view of FIG. 2;

FIG. 4 shows a detail view of a front articulation part;

FIG. 5 shows a detail view of an upper part;

FIG. 6 shows a detail view of a lower part with a fitted artificial lower leg;

FIG. 7 shows a detail view of a hydraulic piston with piston rod;

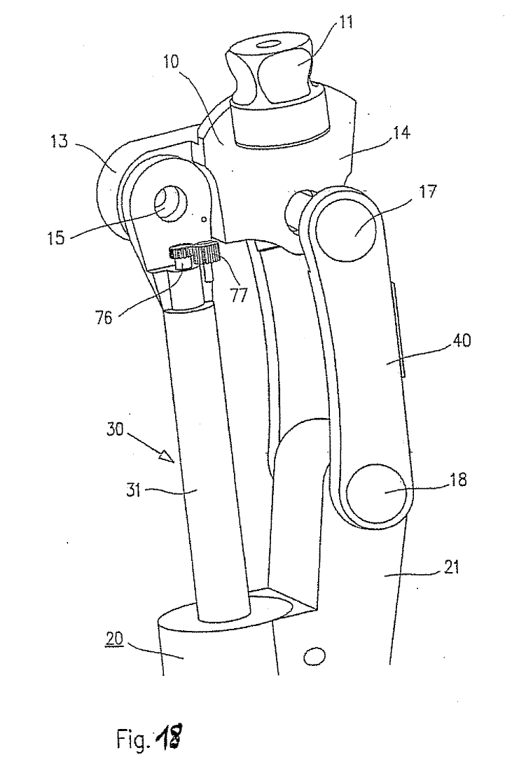

FIG. 8 shows a cross-sectional view of a prosthetic knee joint according to the invention with an articulation device;

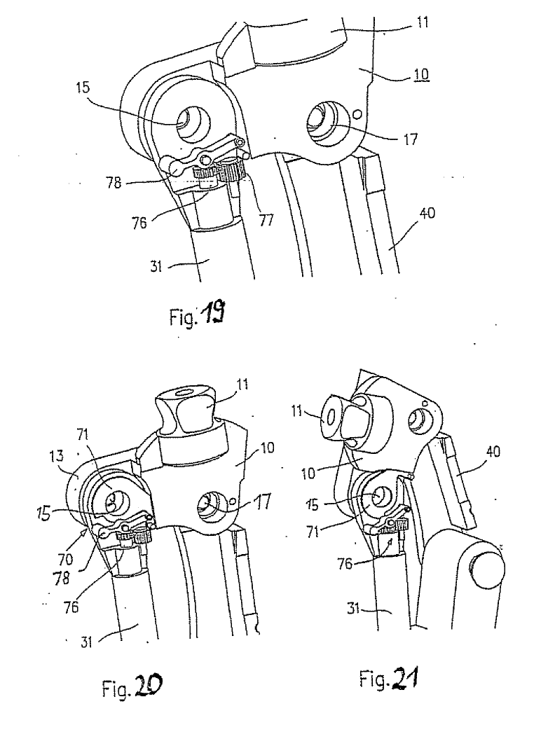

FIG. 9 shows an enlarged view of a resistance device;

FIGS. 10 to 13 show different switch states of a valve system;

FIG. 14 shows a detail view of an auxiliary valve;

FIG. 15 shows a partial cross-sectional view of the valve system;

FIGS. 16 and 17 show an isolated view of a control rod with an adjustment device for the auxiliary valve;

FIG. 18 shows an enlarged view of the prosthetic knee joint with upper part, lower part, the resistance device and the front articulation part;

FIG. 19 shows a detail view of the operating device for the control rod;

FIG. 20 shows a view of a first cam disk in the unswitched state;

FIG. 21 shows a view according to FIG. 20 when the control rod is actuated;

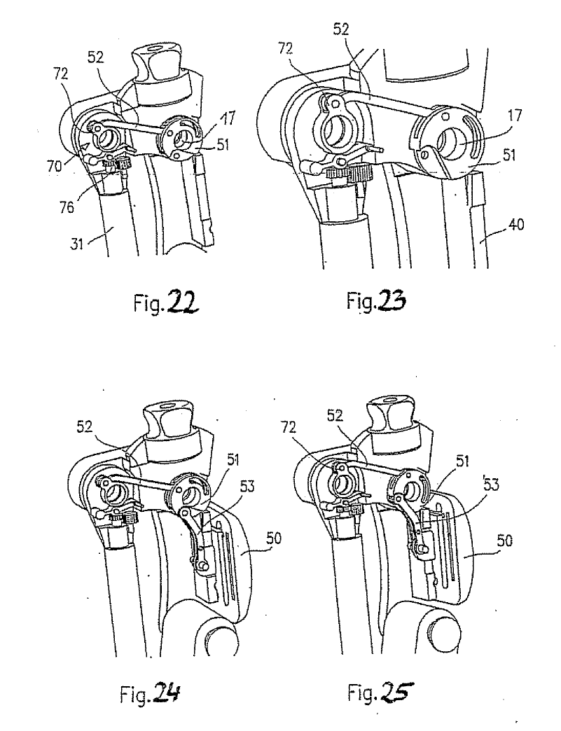

FIGS. 22 and 23 show the arrangement of a second cam disk and a rotary member;

FIG. 24 shows the mechanical coupling of the operating element to the rotary member and the cam disk;

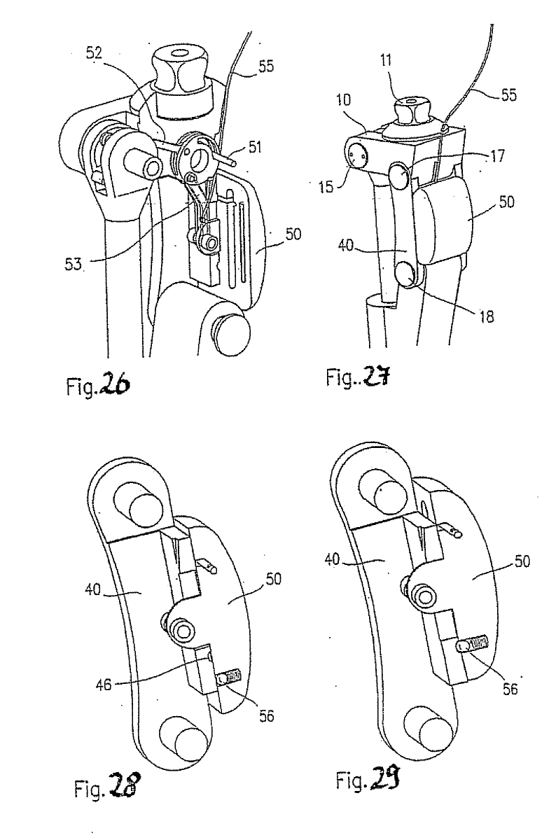

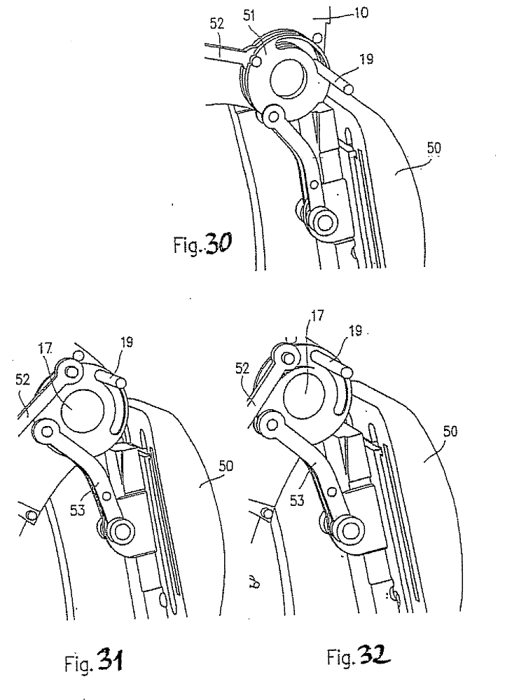

FIG. 25 shows a view according to FIG. 24 in a switched state in which the auxiliary valve is opened;

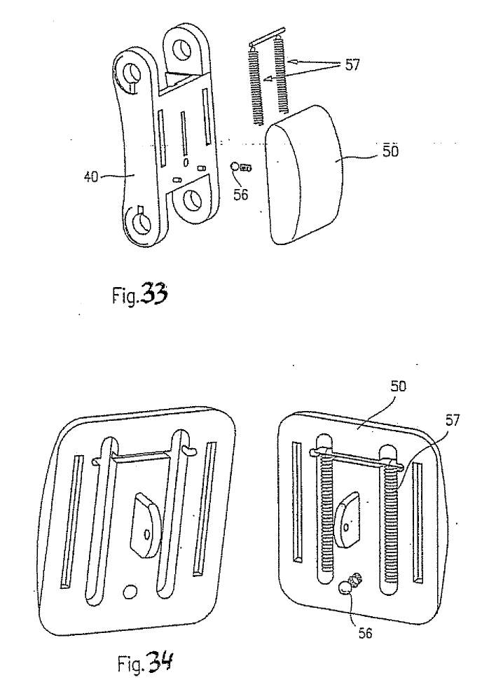

FIGS. 26 and 27 show an arrangement of an operating cable on the operating device;

FIGS. 28 and 29 show sectional views of the operating devices in the locked state and the unlocked state;

FIGS. 30 to 32 show views of the states of the mechanical control as a function of the flexion angle;

FIG. 33 shows an exploded view of the front articulation part and of the operating device;

FIG. 34 shows rear views of the operating device with accessories;

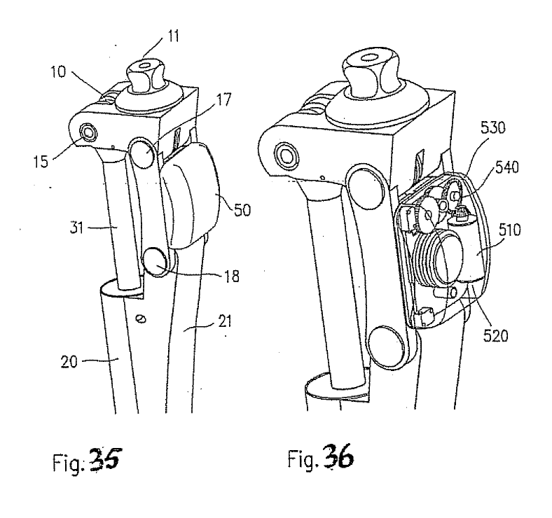

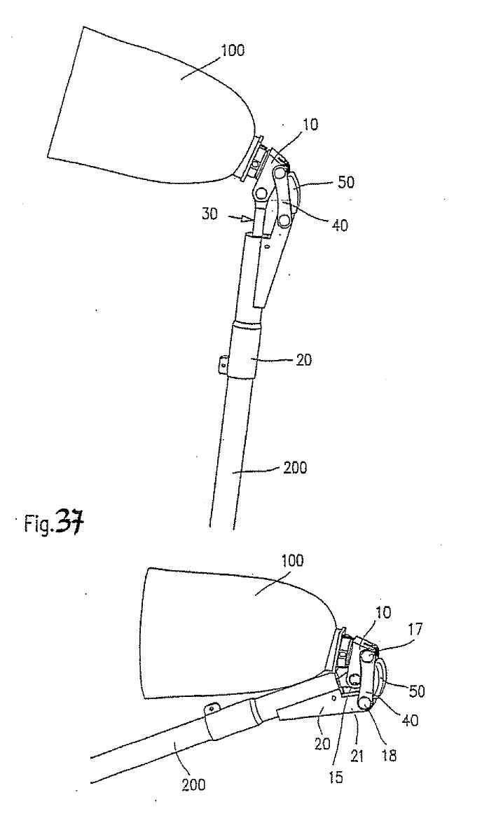

FIGS. 35 and 36 show views of the knee joint with a motor-driven operating device;

FIG. 37 shows two overall views of the prosthetic knee joint in different angle settings; and

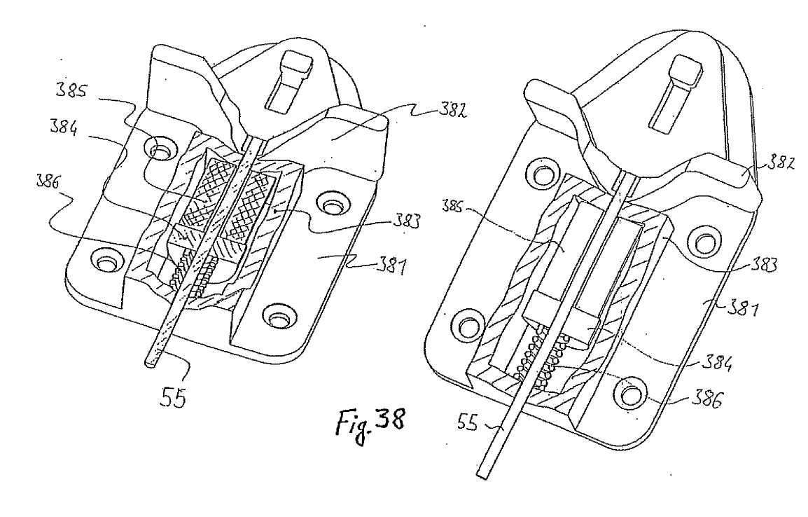

FIG. 38 shows an embodiment of a delay element.

FIG. 1 gives an overall view of a prosthetic knee joint 1 according to the invention which has an upper part 10 and a lower part 20, the upper part 10 and the lower part 20 being connected pivotably to one another via an articulation device. This articulation device comprises a front articulation lever 40 which is secured rotatably both on the upper part 10 and also on the lower part 20. A resistance device 30, which is designed as a hydraulic cylinder device, prevents an uncontrolled flexion movement of the lower part 20 relative to the upper part 10 counter to the normal walking direction, that is to say a forwardly directed walking direction, and it serves at the same time as a rear articulation part. The configuration of the articulation device has the effect that a large part of the load which arises during walking or standing, and which is exerted on the prosthetic knee joint 1 by the prosthesis user, is introduced via the resistance device 30 into the lower part 20 and from there into an artificial lower leg 200.

In order to connect the artificial lower leg 200 to the lower part 20, a corresponding receptacle 25 is formed at the lower end of the lower part 20. Alternatively, the lower part 20 and the artificial lower leg 200 can be designed in one piece and, if appropriate, can be equipped with an artificial foot. At the upper end of the upper part 10 there is a receptacle 100 for the leg stump, the receptacle 100 being able to be secured on the upper part 10 via a fastening device 11, which is shown in FIG. 2.

The prosthetic knee joint 1 further comprises an operating device 50 which is arranged on the front articulation lever 40 and configured like a knee cap. The operating device 50 is mounted displaceably on the front articulation lever 40, and its function will be described further below.

It will be seen from FIG. 2 that the front articulation lever 40 is mounted rotatably on a bearing bracket 21 of the lower part 20. The articulation lever 40 is likewise mounted rotatably on a front section 14 of the upper part 10, whereas the resistance device 30 is arranged rotatably on a rear section 13 of the upper part 10 as seen in the forward direction of walking.

FIG. 3 shows the prosthetic knee joint 1 in a partial cross-sectional view illustrating the configuration of the resistance device 30 with a hydraulic piston 30′ which is connected to the upper part 10 via a piston rod 31. The piston 30′ moves inside a cylinder 26 which is formed by and through the guide 23. The guide 23 is configured as a cylinder wall and forms an integral structural component of the lower part 20. Arranged at the lower end of the cylinder 26 there is a closure device via which the cylinder 26 can be filled with a hydraulic fluid.

Individual parts of the articulation structure are shown in FIGS. 4 to 7, FIG. 4 illustrating the front articulation part 40 which is designed as an articulation lever with two rotation axles. FIG. 5 shows the upper part 10 with the fastening device 11 for the receptacle for a leg stump; the upper part 10 has two recesses for rotation axles in order to be able to receive the resistance device 30 and the front articulation part 40 in a rotatable manner. FIG. 6 shows a lower part 20 with the receptacle 25 for the artificial lower leg 200, said lower part having a substantially tubular configuration on which a front bearing bracket 21 is formed or secured. Together with the lower part 20, the structural component shown in FIG. 7 forms the resistance device 30, said structural component consisting of a piston rod 31 and a piston 30′. At the upper end of the piston rod 31 there is a bore which allows the piston rod 31 to be mounted rotatably in the rear section 13 of the upper part 10.

FIG. 8 shows an alternative structure of the articulation device in which the upper part 10 is articulated directly on the bearing bracket 21, and the piston rod 31 is connected to the rear section 13 of the upper part via a rear articulation lever 12.

FIG. 9 shows a cross section through the lower part 20, so as to illustrate the function of the resistance device. The outer wall 23 of the lower part 20 has a cylindrical configuration and forms a cylinder space 26 in which the piston 30′ is axially displaceably guided. The piston 30′ is designed as a conventional hydraulic piston on which a force acts axially via the piston rod 31. Arranged centrally inside the piston 30′ there is a controllable valve system 60 which is switched via a control rod 76 guided centrally in the piston rod 31. The switching is effected by the valves of the valve system 60 being loaded in the axial direction.

In FIG. 11, the valve system 60 comprises a main valve 61 which is designed as a nonreturn valve and is arranged inside the piston 30′ in such a way that an upward movement of the piston 30′ and thus of the piston rod 31 is at all times possible, but a downward falling movement of the piston 30′ is prevented. For the prosthetic knee joint 1, this means that extension of the knee joint, that is to say a pivoting of the lower part 20 relative to the upper part 10 in the normal walking direction is at all time possible, whereas unwanted flexion and consequent lowering of the prosthesis user counter to the normal walking direction is prevented.

In FIG. 10, the piston 30′ is shown on an enlarged scale. In this state, the control rod 31 does not press the main valve 61 downward, with the result that the valve 61 prevents the downward movement of the piston 30′ but permits an upward movement of the piston 30′, since the hydraulic oil is able to flow unimpeded through the bores 32 from the upper chamber into the lower chamber.

FIG. 11 shows the switched state of the main valve 61, that is to say hydraulic fluid can flow from the lower chamber into the upper chamber through the bores 32, so that a downward movement of the piston 30′ is permitted. In this states the valve 61 allows the piston 30′ to move freely in both directions, which signifies free mobility of the knee joint.

FIG. 12 shows a cross-sectional view also of the valve system 60, clearly illustrating how a similarly configured auxiliary valve 62 is arranged inside the main valve 61. Its function corresponds substantially to that of the main valve 61; in the position according to FIG. 12, the control rod 76 actuates neither the main valve 61 nor the auxiliary valve 62, such that both valves 61, 62 block the downward movement of the piston 30′ and thus block a flexion movement. The main valve 62 permits flow from the upper chamber into the lower chamber, such that extension is possible at any time. It will be seen from FIG. 12 that the control rod 76 has two shoulders 761, 762 which are assigned to the respective valves 61, 62. The shoulders 761, 762 are axially offset relative to one another, the second shoulder 762 coming into engagement with the auxiliary valve 62 earlier than does the first shoulder 761 with the main valve.

FIG. 13 shows a state in which the auxiliary valve 62 is switched via the control rod 31, that is to say is opened. The lower maximum flow rate through the auxiliary valve 62 means that when the auxiliary valve 62 is opened, a downward movement of the piston 30′ and thus a flexion of the knee joint is permitted, but only with a high level of resistance, and this leads to a gentle, decelerated movement and damping, thus permitting controlled lowering of the body from the standing position to the seated position. The flow of liquid is indicated by the arrows and is routed along a valve disk 63, a valve stem 64 and through corresponding bores into the upper cylinder chamber. When the auxiliary valve 62 is not switched, it acts likewise as a nonreturn valve and closes through-opening inside the main valve 61, in which the auxiliary valve 62 is guided via the valve stem 64. In the present illustrative embodiment, the auxiliary valve 62 is arranged concentrically with respect to the main valve 61.

FIG. 14 shows the auxiliary valve 62 on its own, the left-hand side of the figure showing a complete auxiliary valve 62 which has a valve disk 63 and a valve stem 64. The right-hand side of FIG. 14 shows a cross section of the valve stem 64, which has an oval configuration. At the end of the valve 62 remote from the valve disk 63, there is a flattened area, which is designed for rotationally fixed coupling to the control rod 76.

It will be seen from FIG. 15 why the valve stem 64 has a non-rotationally symmetrical cross section. The valve 62 is guided in a round valve guide inside the main valve 61 and, by simple turning of the valve stem 64, it is possible to adjust the flow rate allowed through the auxiliary valve 62, since the flow rate is limited by reducing the cross section of flow. This turning of the auxiliary valve 62 is advantageously effected via the control rod 76, as is shown in FIGS. 16 and 17, namely via an adjusting wheel 77, which moves the control rod 76 in the area where the piston rod is received.

FIG. 18 shows an overall view of the articulation device with the upper part 10, the lower part 20, the front articulation part 40 and the resistance device 30. The front articulation part 40 is mounted rotatably on the upper part 10 and lower part 21 via an upper rotation axle 17 and a lower rotation axle 18, respectively. The piston rod 31 is mounted rotatably on the rear section 13 of the upper part 10 via the rotation axle 15; the piston rod 31 itself is guided axially displaceably inside the lower part 20.

FIG. 19 shows a pivotably mounted pressure lever 78 which is arranged above the end of the control rod 76. In the present illustrative embodiment, the pressure lever 78 is mounted pivotably on the piston rod 31 below the rotation axle 15 and has a cylindrical supporting body whose axis is parallel to the axis of rotation of the pressure lever 78. By this means, precise control of the auxiliary valve 62 and, if appropriate, of the main valve 61 is obtained via a cam disk 71, as is shown in FIG. 20. FIG. 20 shows how the cam disk 71 is connected in a rotationally fixed manner to the upper part 10 and extends above the pressure lever 78, In FIG. 20, the control rod 76 is situated in the upper position, which represents the normal position, since a hydraulic pressure at all times acts counter to the nonreturn valves 61, 62 because of the load that is exerted during standing.

It is only when a defined angle is reached, as is shown in FIG. 21, that a projection formed on the cam disk 71 causes the control rod 76 to be pressed downward so that the auxiliary valve 62 first opens and then the main valve 61. In the illustrative embodiment shown, the upper part 10 has to be pivoted relative to the lower part 20 by an angle of 70° until the first cam disk 71 presses the control rod 76 downward and thus opens the valves 61, 62. In such an articulation position, the knee joint is freely movable, which is advantageous for a prosthesis user in a seated position. The first cam disk 71 is a first element of a control device 70 which, in conjunction with the valve system 60, ensures that a free extension of the lower part 20 is at all times possible and that, above a defined angle range, a free flexion is at all times possible in order to permit comfortable sitting. This also ensures that a locking action counter to the flexion direction is present when the resistance device 30 is not opened and the defined angle range is not exceeded. On the other hand, a controllable and relatively high level of resistance against flexion is possible in an opened state of the auxiliary valve, in order to allow a change from a standing position to a seated position to be made in a safe and controlled manner.

A further component of the control device 70 is shown in FIG. 22 in the form of a second cam disk 72 with which it is possible to operate the control rod 76 in such a way that the auxiliary valve 62 is opened and can thus be switched to the increased resistance mode. The second cam disk 72 likewise acts on the pressure lever 78, but is arranged in FIG. 22 such that the auxiliary valve 62 is not switched. The second cam disk 72 is connected to a rotary member 51 via a lever 52 mounted rotatably on the cam disk 72, which rotary member 51 is mounted on the front rotation axle 17. By turning of the rotary member 51, the second cam disk 72 is turned via the lever 52, and a projection presses the control rod 76 downward and activates the auxiliary valve 62, which is shown in FIG. 23. To set the rotary member 51 in rotation, the operating device 50 in the illustrative embodiment is configured in the shape of a knee cap, which is connected to the rotary member 51 via a further lever 53.

FIG. 24 shows the combination of the displaceable operating device 50 with the levers 52, 53 and the rotary member 51; in the position according to FIG. 24, the operating device 50 is in a lowered position, which means that the rotary member 51 is turned to the maximum extent counterclockwise and, in the lever arrangement here, the second cam disk 72 is likewise turned to the maximum extent counterclockwise. If the operating device 50 is moved upward, as is shown in FIG. 25, the rotary member 51 turns in the clockwise direction, as also does the second cam disk 72. This has the effect that the projection formed on the second cam disk 72 acts on the pressure lever 78 and presses the control rod 76 downward, as a result of which the auxiliary valve 62 is switched, that is to say is opened, and a gentle lowering from the standing position to the seated position is made possible.

To save the prosthesis user the need to grip the knee cap, an operating cable 55 is provided which can be guided upward from the knee, so that by pulling on the operating cable 55, which is arranged on a lever 53 in the present illustrative embodiment, it is possible to set the operating mode in which a gentle flexion is permitted. The operating cable 55 can likewise be arranged on the rotary member or on the operating device 50 itself. The whole knee joint is shown in a plan view in FIG. 27, together with the operating cable 55.

FIGS. 28 and 29 show a sectional view of the operating device 50 which is mounted displaceably on the front articulation part 40 and has a lock element 56 which is spring-loaded and can engage in a recess 46 formed on the front articulation part 40. If the operating device 55 is raised, as is shown in FIG. 29, the lock element 56, here formed as a ball, engages in the recess 46 with a form fit and holds the operating device 50 in the upper position. This means that the auxiliary valve 62 remains opened via the second cam disk 72 and, consequently, with single actuation of the operating device 50, the resistance remains constant for the entire procedure of sitting down. When the operating device 50 is moved downward again, which is made easy on account of the spring mounting of the form-fit element 56, the second cam disk 72 turns counterclockwise and, because of the hydraulic pressure, the auxiliary valve 62 is closed and further lowering and flexion of the knee joint are avoided. The auxiliary valve 62 remains closed in the lowered position of the operating element 50; the levers 52, 53, the rotary member 51 and the control rod 70 form a catch device which prevents a flexion of the knee joint by virtue of stopping the hydraulic flow. The prosthesis user can at all times extend the knee joint, but without the danger of uncontrolled buckling of the articulation. This embodiment can thus be used as an aid for helping geriatric patients when standing up and sitting down.

Once the seated position is reached, in order to bring about a state of the control device 70 in which the patient is able to stand up in a continuous or stepped manner, without the whole body falling back again in the event of inadequate muscle tension in the healthy leg, provision is made that the auxiliary valve 62 is closed when an angle is reached which permits free mobility of the knee joint on account of the opening of the main valve. This is done by means of arranging a carrier 19 on the upper part 10, which carrier turns the rotary member 51 counterclockwise, starting from a defined angle, and in this way, via the lever 53, moves the operating device 50 from the locked, upper position into the unlocked, lower position.

Such a sequence is shown in FIGS. 30 to 32. When the operating device 50 is in a locked state, the rotary member 51 is turned counterclockwise via the carrier 19, above a defined knee angle, and this has the effect that the lever 53 pushes the operating device 50 downward and thereby unlocks the operating device 50. In the illustrative embodiment, the carrier 19 is secured fixedly on the upper part 10 and is guided in a guide of the rotary member 51.

In order to assist the downward movement of the operating device 50 and, upon unlocking of the operating device 50, to cause a corresponding downward movement and the fastest possible closure of the auxiliary valve 62, two restoring springs 57 are provided inside the operating device 50, as are shown in FIGS. 33 and 34. The restoring springs 57 are mounted inside recesses on the back of the operating device 50 and are supported on the front articulation part 40 via corresponding pins. Secure guiding of the operating device 50 is ensured by a central slit in which a projection engages which is formed on the back of the operating device 50.

An alternative embodiment of the operating device 50 is shown in FIGS. 35 and 36, where the operating device 50 is driven by motor and is preferably activated and deactivated by remote control. A motor 510, an energy accumulator 520, a gear 530 and a control unit 540 are provided inside the operating device 50, and the latter can be driven up and down by these means. The remaining mechanical coupling of the operating device via levers 52, 53, rotary member 51 and cam disks 72, 71 is as described above, so that reference is made to the foregoing. By virtue of a modular design and compatibility of the manual and motor-driven operating device 50, a variant can be chosen according to the requirements or indication. Activation of the operating device 50 by remote control, for example by infrared or radio control, can also be used for other lockable knee joints that do not have a means for aiding in standing up and sitting down. The remote control permits unlocking or locking with minimal effort in a posture which appears safest to the prosthesis user, without said user having to grip the knee or having to take one hand away from a walking aid.

FIG. 37 shows two overall views of the prosthetic knee joint at different angle settings, illustrating the compact construction and the large pivoting range of the lower part 20 relative to the upper part 10.

The advantages of the prosthesis according to the invention lie in the fact that by actuating the operating device, whether manually or by motor, the prosthesis user can switch from a locked state of the knee joint to a movable state, the mobility being such that a substantially constant and relatively high level of resistance against flexion is afforded, so that a controlled, gentle and slow movement from standing to sitting is permitted. The device can be operated either in the area of the prosthetic knee joint or, alternatively, by pulling on an operating cable, such that the operating procedure and the switching to the mode with high resistance can be done in an inconspicuous way. By virtue of the locking of the operating device, it is not necessary to keep hold of the actuation knob, the operating device or the operating cable, and instead a single operating maneuver suffices to maintain the set position of high resistance. The locking can be canceled out at any time by the prosthesis user and switched to the mode of increased resistance relative to flexion. Conversely, a locking against flexion of the prosthetic knee joint can be effected at any time by the prosthesis user.

By virtue of the integration of the hydraulic cylinder in the lower part as an integral load-bearing part, the articulation device has the advantage of a compact structure, which not only saves space but also weight. No separate cylinder is needed, and the resistance device in the form of an integrated hydraulic cylinder also serves at the same time as a load-bearing component.

FIG. 38 shows an illustrative embodiment of a delay element 384/385/386 of a catch device which, in the form of a satellite switch, delays the unlocking. FIG. 38 shows an operating cable 55 which is connected to the catch mechanism of the knee joint and which at the end remote from the joint is connected securely to a grip 382. A spring support 384 is likewise securely connected to the operating cable 55. The grip 382 is mounted displaceably on a base plate 381, said base plate 381 forming a housing 383 for receiving both the operating cable 55 and also the spring support 4. A spring device 385, 386 is arranged in the housing 383, the base plate being secured to a loadable structure, for example a shaft of a leg prosthesis. By pulling the operating cable 55 via the grip 382, the rheological spring element 385, for example a highly viscoelastic elastomer, is subjected to compression loading via the spring support 384 and keeps the operating cable 55 in the pulled position for some time after the operating grip 382 has been let go. One or more spring elements 386, if appropriate with different spring properties, assist in the action of the rheological spring element 385 and/or permit advantageous fine-tuning of the function by means of pretensioning or make assembly easier.

Depending on the rheological properties of the spring device 385/386, the operating cable 55 is restored with a time delay. This time delay can be used to allow switching of the knee joint to be done in a manner that is user-friendly and thus also safer for the patient. After pulling the operating cable 55, the hand in question can be used again for a function assisting the movement and the knee joint can thus be unlocked slowly and in a more controlled manner.

1-13. (canceled)

14. A prosthetic knee joint, comprising:
an upper part having a fastening device adapted for a receptacle for a leg stump;
a lower part pivotably connected to the upper part via an articulation device; and
a resistance device having adjustable resistance and configured to act as a lock which, via a mechanical control device and as a function of an angle, blocks a flexion of the articulation device in a flexed position within a definable angle range,
wherein the lower part is freely pivotable in the flexion direction outside the definable angle range without action of the resistance device.

15. A prosthetic knee joint comprising:
an upper part having a fastening device adapted for a receptacle for a leg stump;
a lower part pivotably connected to the upper part via an articulation device;
a catch device configured to lock the prosthetic knee joint in an extended position, the catch device being configured to be locked and unlocked by an operating device; and
a resistance device with adjustable resistance, the resistance device providing resistance to flexion of the articulation device as a function of an angle within an angle range definable via a mechanical control device,
wherein the lower part is freely pivotable in the flexion direction outside the definable angle range without action of the resistance device.

16. The prosthetic knee joint as claimed in claim 14, wherein the lower part is freely extended.

17. The prosthetic knee joint as claimed in claim 14, wherein the resistance device is configured to increase the resistance to the flexion to a locking action, and the resistance device is configured such that it can be switched.

18. The prosthetic knee joint as claimed in claim 14, wherein at least one of the resistance device and a catch device is coupled to an operating device via which the resistance is increased or decreased or the locking is released or locked.

19. The prosthetic knee joint as claimed in claim 18, wherein the operating device is driven by hand or by motor.

20. The prosthetic knee joint as claimed in claim 19, wherein the operating device is a remote control device.

21. The prosthetic knee joint as claimed in claim 14, wherein the resistance device is connected to a mechanical control device which has at least one cam disk and switches the resistance device as a function of the angle of flexion of the upper part relative to the lower part.

22. The prosthetic knee joint as claimed in claim 14, wherein the resistance device is a hydraulic or pneumatic unit, a friction coupling or an electromagnetic coupling, or a magnetorheological or piezoelectric device.

23. The prosthetic knee joint as claimed in claim 22, wherein the hydraulic or pneumatic unit has a controllable valve system which is arranged inside a piston guided in a cylinder.

24. A prosthetic knee joint, comprising;
an upper part having a fastening device for a receptacle for a leg stump;
a lower part pivotably connected to the upper part via an articulation device;
a catch device configured to lock the prosthetic knee joint in an extended position, the catch device being configured to be locked and unlocked by an operating device, the operating device being operated by remote control.

25. A prosthetic knee joint, comprising:
an upper part which has a fastening device for a receptacle for a leg stump;
a lower part pivotably connected to the upper part via an articulation device;
a catch device configured to lock the prosthetic knee joint in the extended position, the catch device being locked and unlocked by an operating device; and
a delay element associated with the catch device which unlocks or re-locks the catch device after a time delay after activation of the unlocking.

26. The prosthetic knee joint as claimed in claim 25, wherein the delay element is a relay, an elastic and/or Theological element or an electronic circuit with actuator.

27. The prosthetic knee joint as claimed in claim 15, wherein the lower part is freely extended.

28. The prosthetic knee joint as claimed in claim 15, wherein the resistance device is configured to increase the resistance to the flexion to a locking action, and the resistance device is configured such that it can be switched.

29. The prosthetic knee joint as claimed in claim 15 wherein at least one of the resistance device and the catch device is coupled to an operating device via which the resistance is increased or decreased or the locking is released or locked.

30. The prosthetic knee joint as claimed in claim 29, wherein the operating device is driven by hand or by motor.

31. The prosthetic knee joint as claimed in claim 30, wherein the operating device is a remote control device.

32. The prosthetic knee joint as claimed in claim 15, wherein the resistance device is connected to a mechanical control device which has at least one cam disk and switches the resistance device as a function of the angle of flexion of the upper part relative to the lower part.

33. The prosthetic knee joint as claimed in claim 15, wherein the resistance device is a hydraulic or pneumatic unit, a friction coupling or an electromagnetic coupling, or a magnetorheological or piezoelectric device.

34. The prosthetic knee joint as claimed in claim 33, wherein the hydraulic or pneumatic unit has a controllable valve system which is arranged inside a piston guided in a cylinder.
